SEQUENCE LISTING (2 sequences)

SEQ ID NO: 1 - DNA, 198 bp, Artificial Sequence
  synthesized
gcccatgaacacgagcacgaggcggaaaacaaattcaacaaagaaatgcgcaacgcgtactgggaaattgccctgctgccgaacctgaccaaccaacagaaacgcgccttcatccgctccctgtacgacgacccatcccaatctgcaaacctgctggcggaagcgaagaaactgaacgatgcacagggtggtggttgc

SEQ ID NO: 2 - protein, 66 aa, Artificial Sequence
  synthesized
AHEHEHEAENKFNKEMRNAYWEIALLPNLTNQQKRAFIRSLYDDPSQSANLLAEAKKLNDAQGGGC

(19) United States
(12) Patent Application Publication
(10) Pub. No.: US 2024/0207460 A1
(43) Pub. Date: Jun. 27, 2024

(54) HER2 AFFIBODY 99MTC MARKER COMPOSITION AND APPLICATION THEREOF

(71) Applicant: JIANGSU YUANBEN BIOTECHNOLOGY CO., LTD., Taizhou, Jiangsu (CN)

(72) Inventors: Jiong CAI, Taizhou, Jiangsu (CN); Yi LIU, Taizhou, Jiangsu (CN); Dalin GAO, Taizhou, Jiangsu (CN); Xu SONG, Taizhou, Jiangsu (CN); Kai XIAO, Taizhou, Jiangsu (CN); Chen LIANG, Taizhou, Jiangsu (CN); Guozhong DING, Taizhou, Jiangsu (CN); Mingxia LIU, Taizhou, Jiangsu (CN)

(21) Appl. No.: 18/559,294

(22) PCT Filed: Aug. 16, 2021
(86) PCT No.: PCT/CN2021/112782

§ 371 Date: Nov. 6, 2023

(30) Foreign Application Priority Data
Mar. 3, 2021 (CN) 202110234038.5

(51) Int. Cl. A61K 51/10; A61K 47/02; A61K 47/12; A61K 47/18; A61K 47/22; C07K 16/32

(52) U.S. Cl. CPC A61K 51/103; A61K 47/02; A61K 47/12; A61K 47/183; A61K 47/22; C07K 16/32; A61K 2123/00

(57) ABSTRACT

An HER2 affibody ^{99m}Tc marker composition and application thereof are provided, which relates to the field of radiopharmaceuticals and nuclear medicine. The HER2 affibody ^{99m}Tc marker composition contains HEPES, sodium glucoheptonate, vitamin C, cysteine, stannous chloride and an HER2 affibody.

### TECHNICAL FIELD

The present disclosure relates to the fields of radiopharmaceuticals and nuclear medicine and in particular to an HER2 affibody ^{99m}Tc labelling composition and application thereof.

### BACKGROUND

Human Epidermal Growth Factor Receptor 2 (HER2) is a transmembrane protein and plays an important role in cell growth, survival and differentiation. About ⅓ of breast cancer patients have significantly increased HER2 expression, and the cells with high HER2 expression exhibit abnormal proliferation, which finally leads to tumor generation. HER2 expression-positive breast cancer (HER2+BC) is the most aggressive subtype of breast cancer. It often has poor prognosis and is susceptible to recurrence, and the probability of its resistance to chemotherapy and endocrine therapy is greatly increased. Therefore, HER2 is an important molecular imaging marker of HER2+BC and an ideal subject for targeted therapy. There are two important challenges for HER2 targeted therapy. One is the screening of patients with high expression of HER2; only patients with high expression of HER2 may benefit from targeted therapy, whereas patients with no or low expression of HER2 may be over-treated, potentially increasing the side effects of targeted therapy and missing a more suitable treatment regimen. The other one is the monitoring of the treatment effect; there is a need to find a dynamic, noninvasive and integral method for evaluating targeted therapy so as to achieve the optimal therapeutic effect.

Clinically, what is usually adopted for detecting the HER2 expression of tumors is to take a biopsy specimen for biochemical and histopathological examinations, which involve the detection of the level of HER2 gene by using the real-time quantitative PCR, the detection of the expression quantity of HER2 protein by using the western blot electrophoresis, or the detection of the expression of HER2 protein of a pathological tissue section by using the immunochemical staining. Although pathological biopsy is a gold standard for evaluating HER2 expression, such an invasive examination cannot be performed many times as a routine examination for tumor efficacy evaluation, and the accuracy depends on the clinical experience and level of the operator. Besides, the local condition of biopsy specimen cannot reflect the condition of the whole tumor, and therefore the pathological biopsy is lack of timeliness and comprehensiveness. The CT and MRI often analyze tumor characteristics and treatment response based on anatomical morphological features and lack the analysis of the expression of the underlying biological and physiological information in tumors, especially lack the sensitivity to changes in the biological and physiological information during the initial period of treatment. For the monitoring of the curative effect of the molecular targeted drug, the CT and MRI are also faced with challenges, because the molecular targeted drug usually has an inhibition effect on tumor cells and the morphological characteristics of the tumor are not significantly changed. To get the correlation between the extent of survival benefit and toxic effects as well as the pharmaceutical effect in the molecular targeted therapy necessitates a more accurate and rapid method for evaluating HER2 status in clinical practice. Therefore, it is a key and difficult point of HER2+BC diagnosis and treatment to find a method for in vivo, noninvasive, integral and dynamic evaluation of HER2 expression detection.

HER2 imaging has the following significances: firstly, screening the most appropriate treatment regimen by imaging; secondly, performing early judgement and prediction for the treatment effect through imaging; thirdly, combining the diagnosis and treatment drug through a carrier. Diagnosis and treatment integration is a new trend of nuclear medicine research and development in recent years, and treatment is guided according to individual conditions of patients on the basis of nuclide imaging, so that adverse effects are reduced, and the treatment effect is improved. Nuclide imaging becomes an important means of the diagnosis and treatment integration by detecting the changes of the function of organs in vivo, and the molecular probe that is based on the diagnosis and treatment integration is used for detecting the changes of the physiological function of diseases and simultaneously implementing the treatment function. Because the characteristic of HER2+BC of highly expressing HER2 receptor and the prediction of curative effect of and the drug resistance of current molecular targeted therapy are still the key problems in clinical practice, the development of a diagnosis and treatment integrated molecular probe based on HER2 receptor is an important strategy for realizing accurate diagnosis and treatment of breast cancer.

An affibody is a novel scaffold protein originally derived from Staphylococcus aureus protein A. It consists of 58 amino acid residues and has a molecular weight between that of an antibody and that of a polypeptide (about 6.5 kDa). It can specifically bind to a target protein and has high affinity and target specificity. The size of the binding region for an antigen of an affibody is equivalent to that of an antibody. The affibody has no activating effect, so there may not be too much limitation on the injection dose thereof. Because the affibody has the advantages of small relative molecular weight, high binding affinity, strong specificity, rapid targeted aggregation, rapid blood clearance and the like, the radiolabeled HER2-targeted affibody has good application value in the imaging of HER2 expression-positive tumors in vivo.

Li Xin et al. successfully prepared a ⁹⁹Tc^{m}-human epidermal growth factor receptor 2 (HER2) affibody (ABH2) and demonstrated that the ⁹⁹Tc^{m}-ABH2 can specifically image the HER2-positive breast cancer through a tumor-bearing mouse experiment. On the premise of ensuring tumor targeting ability, the radiation damage to non-tumor tissues is reduced as much as possible, and the non-specific uptake of the liver is reduced to improve the detection efficiency for a focus, the conversion of basic research to clinical application is accelerated, individualized diagnosis and treatment are promoted, and the important strategy of early discovery and early treatment of HER2+BC is realized (Li Xin, Cai Jiong, Zhu Zhaohui, Li Fang. Preparation of HER2 radioligand ^{99m}Tc-ABH2 and its in vivo imaging in breast cancer-xenografted mice. Chinese Journal of Nuclear Medicine and Molecular Imaging. 2015, 35(3): 222-226). Ahlgrena et al. carried out pre-clinical pharmaceutical formula research on a small-molecule HER2-targeted single photon imaging affibody radiopharmaceutical, and the components of kit are glucoheptonic acid, EDTA and stannous chloride, but the formula has the problem of low labeling rate, which is less than 10%. In contrast, the labeling rate of the formula consisting of glucoheptonic acid, HEPES and stannous chloride is around 90% (Tait J. F., Brown D. S., Gibson D. F., Blankenberg F. G., Strauss H. W. Development and characterization of annexin V mutants with endogenous chelation sites for (99m)Tc. Bioconjug Chem. 2000, 11(6):918-925). Even so, there is the problem of short shelf life, because kits need to be preserved at a low temperature, usually at the temperature of −20° C., and their shelf life is about 1 month, and the labeling rate of a kit stored at the temperature of −20° C. for 3 months is only about 80%.

Therefore, there is an urgent need to develop a more stable HER2 affibody ^{99m}Tc labelling composition for HER2 imaging.

### SUMMARY

In order to achieve the above object, the present disclosure provides the following technical schemes:

In a first aspect, the present disclosure provides an HER2 affibody ^{99m}Tc labelling composition, comprising: HEPES, sodium glucoheptonate, vitamin C, cysteine, stannous chloride and an HER2 affibody.

In a specific embodiment of the present disclosure, wherein the HEPES has a concentration of 2-30 mmol/L, the sodium glucoheptonate has a concentration of 5-40 mmol/L, the vitamin C has a concentration of 2-30 mmol/L, the cysteine has a concentration of 5-40 mmol/L, the stannous chloride has a concentration of 0.1-5 mmol/L, and the HER2 affibody has a concentration of 0.01-0.6 mmol/L, and the composition has a pH of 5.0-7.5.

In a specific embodiment of the present disclosure, wherein the HEPES has a concentration of 5-20 mmol/L, the sodium glucoheptonate has a concentration of 10-30 mmol/L, the vitamin C has a concentration of 5-20 mmol/L, the cysteine has a concentration of 10-30 mmol/L, the stannous chloride has a concentration of 0.2-2 mmol/L, and the HER2 affibody has a concentration of 0.03-0.3 mmol/L, and the composition has a pH of 6.0-7.0.

In a specific embodiment of the present disclosure, wherein the HEPES has a concentration of 10 mmol/L, the sodium glucoheptonate has a concentration of 20 mmol/L, the vitamin C has a concentration of 10 mmol/L, the cysteine has a concentration of 20 mmol/L, the stannous chloride has a concentration of 0.4 mmol/L, and the HER2 affibody has a concentration of 0.05 mmol/L, and the composition has a pH of 6.6.

In a specific embodiment of the present disclosure, wherein the HER2 affibody has GGGC at carboxyl terminus for isotope labeling and HEHEHE affinity column purification at amino terminus. In another aspect, the present disclosure provides use of the HER2 affinity ^{99m}Tc labelling composition described in the first aspect of the present disclosure in the preparation of an imaging agent product.

In another aspect, the present disclosure provides an imaging agent product, comprising the HER2 affibody ^{99m}Tc labelling composition described in the first aspect of the present disclosure.

In a specific embodiment of the present disclosure, the imaging agent is a PET or SPECT imaging agent and the product is a separate agent or a kit.

In another aspect, the present disclosure provides a method for preparing an HER2 affibody ^{99m}Tc labelling composition, comprising the following steps: adding 500-20000 μCi of ^{99m}Tc into the HER2 affibody ^{99m}Tc labelling composition described in the first aspect of the present disclosure. In a specific embodiment of the present disclosure, wherein the ^{99m}Tc is added in an amount of 2000-10000 μCi, preferably 5000 μCi.

In a specific embodiment of the present disclosure, the reaction time is 10-15 min.

Compared with the prior art, the present disclosure has the following beneficial effects:

The HER2 affibody ^{99m}Tc labelling composition described in the present disclosure has a high labeling rate, is not prone to generate dimers, has good stability and can be preserved for no less than 60 days at room temperature, which reduces storage cost and transportation cost and allows it to have better clinical application prospect.

### BRIEF DESCRIPTION OF THE DRAWINGS

FIG. 1 is a graph showing the results of dimer identification by SDS-PAGE after 60 days of preservation at a low temperature and at room temperature;

FIG. 2 is a graph showing the results of time limits of preservation of HER2 affibody in different formulas;

FIG. 3 is a graph showing the results of the stability tests of the HER2 affibody-expressing Escherichia coli supernatant and the HER2 affibody-expressing Escherichia coli bacterial cells;

FIG. 4 is a group of graphs showing the ITLC detection results of ^{99m}Tc-ABH2 in Experimental Examples 2-10;

### DETAILED DESCRIPTION

The present disclosure will be described in further detail with reference to specific examples, which are not intended to limit the present disclosure but only to illustrate the present disclosure. Unless otherwise stated, the experimental methods used in the following examples, and the experimental methods without specific conditions indicated in the examples are generally performed under conventional conditions. Unless otherwise stated, the materials, reagents and the like used in the following examples are commercially available.

Unless otherwise defined, all technical and scientific terms used herein have the meaning commonly understood by those of skill in the art.

### Example 1. Preparation of HER2 Affibody (ABH2)

The carboxyl terminus of the affibody was designed with a GGGC sequence for isotope labeling, and the amino terminus was designed with an HEHEHE sequence for affinity column purification. An HER2 affibody gene (SEQ ID NO: 1) with a preference for Escherichia coli expression was designed by using DNAstar software according to a triple code, the HER2 affibody gene was then synthesized by the oligonucleotide synthesis and the overlap PCR method, the enzyme digestion by HindIII was carried out to identify the length of the synthesized gene, and the sequence of the synthesized gene was determined by gene sequencing.

The HER2 affibody gene was digested with enzymes Ncol and EcoRI, inserted into a pET22b(+) vector subjected to enzyme digestion in the same way, and then transformed into DH5a competent Escherichia coli. The bacterial liquid was sent for gene sequencing identification after screening with antibiotics, and a plasmid was extracted and transformed into BL21(DE3) competent Escherichia coli. The expression was induced with IPTG, and bacterial clones expressing the nascent protein were identified by SDS-PAGE.

The Escherichia coli expressing the HER2 affibody was centrifuged, and the bacterial cells were washed with a washing buffer (50 mM Na₂HPO₄, 300 mM NaCl, pH 7.0), resuspended in a purification buffer (20 mM Tris HCl, 500 mM NaCl, pH 7.9) and crushed by ultrasonication. Cell debris was removed by centrifugation and the supernatant was treated at 60° ° C. for 10 min. Then the supernatant was centrifuged, filtered through a filter membrane and applied to a nickel column equilibrated in advance with a crushing buffer (20 mM Tris HCl, 500 mM NaCl, 1 mM EDTA, pH 7.9). After washing with a purification buffer containing 5 mM imidazole, purification was performed by eluting with a purification buffer containing 60 mM imidazole. Then the ion exchange purification was performed through a Q column, and the purification was carried out under the condition of 0.5 M NaCl. The product obtained after purification was subjected to ultrafiltration to remove the buffer and replace it with an aqueous solution, thus obtaining the HER2 affibody-expressing Escherichia coli bacterial cells.

### Example 2

The difference from Example 1 is that after the Escherichia coli expressing the HER2 affibody was centrifuged, the supernatant was directly dialyzed against a purification buffer (20 mM Tris HCl, 500 mM NaCl, pH 7.9) overnight and then centrifuged, and the supernatant was filtered through a filter membrane and applied to a nickel column equilibrated in advance with a crushing buffer. After washing with a purification buffer containing 5 mM imidazole, purification was performed by eluting with a purification buffer containing 60 mM imidazole. Then the ion exchange purification was performed through a Q column, and the purification was carried out under the condition of 0.5 M NaCl. The product obtained after purification was subjected to ultrafiltration to remove the buffer and replace it with an aqueous solution, thus obtaining the HER2 affibody-expressing Escherichia coli supernatant.

### Example 3

HER2 Affibody ^{99m}Tc Labelling ^{99m}Tc was used to label the HER2 affibody, and the specific steps are as follows: vitamin C was added to the affibody prepared in Example 1 or 2, and the concentration of the affibody was adjusted to 1 mg/mL. 100 μL of the affibody after the concentration adjustment was added into a labeling bottle, 200 μL of deoxidized labeling buffer (10 mmol/L of HEPES, 20 mmol/L of sodium glucoheptonate, 20 mmol/L of cysteine, 0.4 mmol/L of stannous chloride) was added, and then 5000 μCi of ^{99m}Tc was added. The mixture was reacted at room temperature for 10 min to complete the reaction.

The ITLC detection showed that the ^{99m}Tc-ABH2 was significantly different from the ^{99m}Tc, the Rf of the ^{99m}Tc-ABH2 was 0, and the Rf of the ^{99m}Tc was 1.

### Experimental Example 1: Stability Test

1. The purified HER2 affibody in Example 1 was added to different composition formulas and preserved at room temperature and a low temperature of 4° C. separately, and dimer production was identified by SDS-PAGE every 2-3 days. The composition formulas are shown in Table 1 below.

TABLE 1
Composition formulas

| No. | Conditions | Composition formula |
|---|---|---|
| 1 | Low temperature | 10 mmol/L of HEPES, 20 mmol/L of sodium glucoheptonate |
| | (LT) | |
| 2 | Low temperature | 10 mmol/L of HEPES, 20 mmol/L of sodium glucoheptonate, 0.4 mmol/L |
| | (LT) | of stannous chloride |
| 3 | Low temperature | 10 mmol/L of HEPES, 20 mmol/L of sodium glucoheptonate, argon |
| | (LT) | |
| 4 | Low temperature | 10 mmol/L of HEPES, 20 mmol/L of sodium glucoheptonate, 0.4 mmol/L |
| | (LT) | of stannous chloride, argon |
| 5 | Low temperature | 10 mmol/L of HEPES, 20 mmol/L of sodium glucoheptonate, 20 mmol/L |
| | (LT) | of cysteine |
| 6 | Low temperature | 10 mmol/L of HEPES, 20 mmol/L of sodium glucoheptonate, 10 mmol/L |
| | (LT) | of vitamin C |
| 7 | Low temperature | 10 mmol/L of HEPES, 20 mmol/L of sodium glucoheptonate, 10 mmol/L |
| | (LT) | of vitamin C, 20 mmol/L of cysteine |
| 8 | Low temperature | 10 mmol/L of HEPES, 20 mmol/L of sodium glucoheptonate, 10 mmol/L |
| | (LT) | of vitamin C, 20 mmol/L of cysteine, 0.4 mmol/L of stannous chloride |
| 9 | Room | 10 mmol/L of HEPES, 20 mmol/L of sodium glucoheptonate |
| | temperature (RT) | |
| 10 | Room | 10 mmol/L of HEPES, 20 mmol/L of sodium glucoheptonate, 0.4 mmol/L |
| | temperature (RT) | of stannous chloride |
| 11 | Room | 10 mmol/L of HEPES, 20 mmol/L of sodium glucoheptonate, argon |
| | temperature (RT) | |
| 12 | Room | 10 mmol/L of HEPES, 20 mmol/L of sodium glucoheptonate, 0.4 mmol/L |
| | temperature (RT) | of stannous chloride, argon |
| 13 | Room | 10 mmol/L of HEPES, 20 mmol/L of sodium glucoheptonate, 20 mmol/L |
| | temperature (RT) | of cysteine |
| 14 | Room | 10 mmol/L of HEPES, 20 mmol/L of sodium glucoheptonate, 10 mmol/L |
| | temperature (RT) | of vitamin C |
| 15 | Room | 10 mmol/L of HEPES, 20 mmol/L of sodium glucoheptonate, 10 mmol/L |
| | temperature (RT) | of vitamin C, 20 mmol/L of cysteine |
| 16 | Room | 10 mmol/L of HEPES, 20 mmol/L of sodium glucoheptonate, 10 mmol/L |
| | temperature (RT) | of vitamin C, 20 mmol/L of cysteine, 0.4 mmol/L of stannous chloride |

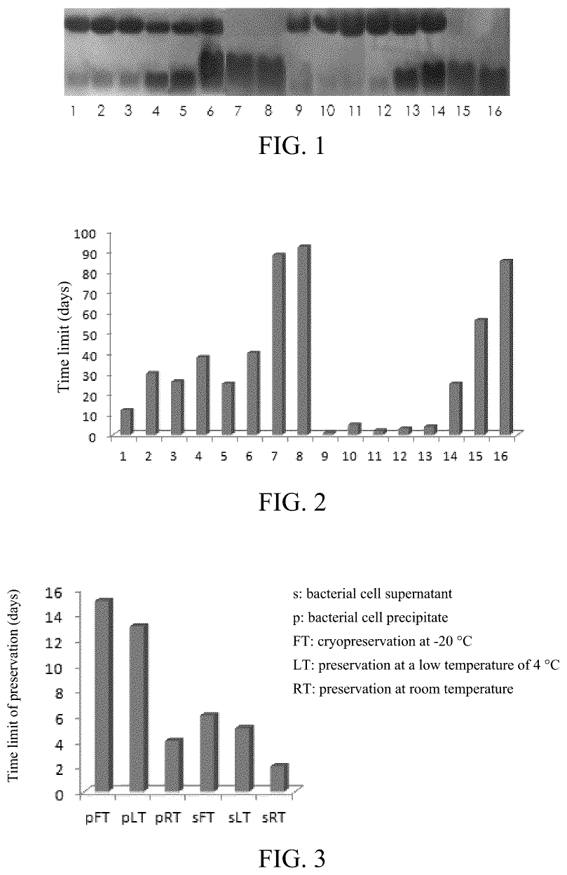

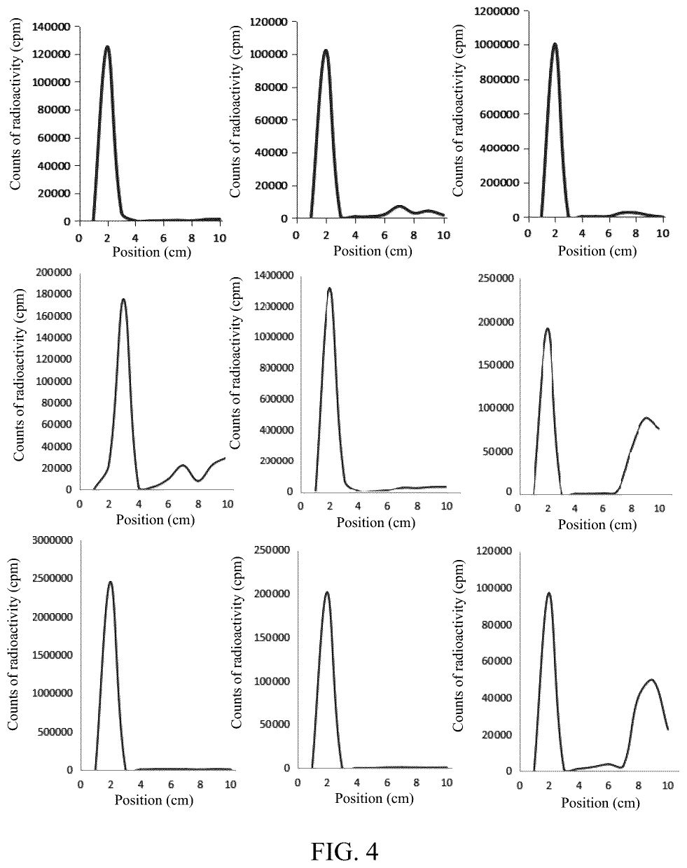

FIG. 1 is a graph showing the results of dimer identification by SDS-PAGE after 60 days of preservation at a low temperature and at room temperature, and FIG. 2 is a graph showing the time limit of storage of HER2 affibody in different formulas.

As can be seen from FIG. 1, the HER2 affibody had poor stability and was easy to generate dimers when vitamin C and cysteine were not added; the stability when cysteine was added was poorer than that when vitamin C was added; the stability when both vitamin C and cysteine were added was better than that when vitamin C or cysteine was added alone.

As can be seen from FIG. 2, the formulas showing better preservation conditions when preserved at a low temperature involved addition of vitamin C and SnCl₂, introduction of argon and the like; HER2 affibody could be preserved for more than 60 days at room temperature when cysteine, vitamin C and SnCl₂ were all added.

2. The purified HER2 affibody in Example 2 was preserved at room temperature, at a low temperature of 4° C. and at a freezing condition of −20° C. separately, and the results are shown in FIG. 3. As can be seen from FIG. 3, the stability of the HER2 affibody-expressing Escherichia coli supernatant was lower than that of the HER2 affibody-expressing Escherichia coli bacterial cells.

### Experimental Example 2

Composition formula: 10 mmol/L of HEPES; 20 mmol/L of sodium glucoheptonate; 10 mmol/L of vitamin C; 20 mmol/L of cysteine; 0.4 mmol/L of stannous chloride; 0.05 mmol/L of HER2 affibody; pH 6.6.

### Isotope: 5000 μCi (463 GBq/L).

In the formula, the HER2 affibody was synthesized as described in Example 1, and ^{99m}Tc-ABH2 was synthesized as described in Example 3.

### Experimental Example 3

Composition formula: 10 mmol/L of HEPES; 20 mmol/L of sodium glucoheptonate; 1 mmol/L of vitamin C; 20 mmol/L of cysteine; 0.4 mmol/L of stannous chloride; 0.05 mmol/L of HER2 affibody; pH 6.6.

### Isotope: 5000 μCi (463 GBq/L).

In the formula, the HER2 affibody was synthesized as described in Example 1, and ^{99m}Tc-ABH2 was synthesized as described in Example 3.

### Experimental Example 4

Composition formula: 10 mmol/L of HEPES; 20 mmol/L of sodium glucoheptonate; 35 mmol/L of vitamin C; 20 mmol/L of cysteine; 0.4 mmol/L of stannous chloride; 0.05 mmol/L of HER2 affibody; pH 6.6.

### Isotope: 5000 μCi (463 GBq/L).

In the formula, the HER2 affibody was synthesized as described in Example 1, and ^{99m}Tc-ABH2 was synthesized as described in Example 3.

### Experimental Example 5

Composition formula: 10 mmol/L of HEPES; 20 mmol/L of sodium glucoheptonate; 10 mmol/L of vitamin C; 4 mmol/L of cysteine; 0.4 mmol/L of stannous chloride; 0.05 mmol/L of HER2 affibody; pH 6.6.

### Isotope: 5000 μCi (463 GBq/L).

In the formula, the HER2 affibody was synthesized as described in Example 1, and ^{99m}Tc-ABH2 was synthesized as described in Example 3.

### Experimental Example 6

Composition formula: 10 mmol/L of HEPES; 20 mmol/L of sodium glucoheptonate; 10 mmol/L of vitamin C; 45 mmol/L of cysteine; 0.4 mmol/L of stannous chloride; 0.05 mmol/L of HER2 affibody; pH 6.6.

### Isotope: 5000 μCi (463 GBq/L).

In the formula, the HER2 affibody was synthesized as described in Example 1, and ^{99m}Tc-ABH2 was synthesized as described in Example 3.

### Experimental Example 7

Composition formula: 10 mmol/L of HEPES; 20 mmol/L of sodium glucoheptonate; 10 mmol/L of vitamin C; 20 mmol/L of cysteine; 0.4 mmol/L of stannous chloride; 0.005 mmol/L of HER2 affibody; pH 6.6.

### Isotope: 5000 μCi (463 GBq/L).

In the formula, the HER2 affibody was synthesized as described in Example 1, and ^{99m}Tc-ABH2 was synthesized as described in Example 3.

### Experimental Example 8

Composition formula: 10 mmol/L of HEPES; 20 mmol/L of sodium glucoheptonate; 10 mmol/L of vitamin C; 20 mmol/L of cysteine; 0.4 mmol/L of stannous chloride; 0.65 mmol/L of HER2 affibody; pH 6.6.

### Isotope: 5000 μCi (463 GBq/L).

In the formula, the HER2 affibody was synthesized as described in Example 1, and ^{99m}Tc-ABH2 was synthesized as described in Example 3.

### Experimental Example 9

Composition formula: 10 mmol/L of HEPES; 20 mmol/L of sodium glucoheptonate; 10 mmol/L of vitamin C; 20 mmol/L of cysteine; 0.4 mmol/L of stannous chloride; 0.05 mmol/L of HER2 affibody; pH 6.6.

### Isotope: 300 μCi (28 GBq/L).

In the formula, the HER2 affibody was synthesized as described in Example 1, and ^{99m}Tc-ABH2 was synthesized as described in Example 3.

### Experimental Example 10

Composition formula: 10 mmol/L of HEPES; 20 mmol/L of sodium glucoheptonate; 10 mmol/L of vitamin C; 20 mmol/L of cysteine; 0.4 mmol/L of stannous chloride; 0.05 mmol/L of HER2 affibody; pH 6.6.

### Isotope: 25000 μCi (2313 GBq/L).

In the formula, the HER2 affibody was synthesized as described in Example 1, and ^{99m}Tc-ABH2 was synthesized as described in Example 3.

### Experimental Example 11

1. Detection of labeling rate: 0.5 μL of the reaction mixture was spotted onto the origin spots of ITLC-SG chromatography paper, and the ascending chromatography was performed using 0.1 M citric acid as the mobile phase. The radioactive counts were measured by a radioactive scanner to calculate the labeling rate.

2. Detection of radio-chemical purity: The ^{99m}Tc-ABH2 was purified by using a Waters Sep-pek solid-phase extraction column and a C18 reverse-phase column, 0.5 μL of the reaction mixture was spotted onto the origin spots of ITLC-SG chromatography paper, and the ascending chromatography was performed using 0.1 M citric acid as the mobile phase. The radioactive counts were measured by a radioactive scanner to calculate the radiochemical purity.

3. Detection of in vitro stability: the ^{99m}Tc-ABH2 prepared in Experimental Examples 2-10 was mixed with 0.1 mol/L of PBS (pH 7.4) or healthy human serum at a volume ratio of 1:10, and the mixture was incubated at 37° C., and the radiochemical purity of the ^{99m}Tc-ABH2 was determined 6 hours later.

4. Detection of biological binding activity: the ovarian cancer cell strain SKOV-3 was cultured in a DMEM high-glucose medium containing 10% FBS (v/v), 100 U/mL of penicillin and 100 μg/mL of streptomycin. SKOV-3 cells were plated in a 24-well plate at 1 mL per well (5×10⁵/mL) 1 day prior to activity assay. On day 2, the ^{99m}Tc-ABH2 was obtained by labeling on site, and the ^{99m}Tc-ABH2 was diluted at a ratio of 1:2 and added at various concentrations (26, 56, 112, 225, 450, 900, 1800 and 36000 nmol/L, 3 parallel wells per concentration)(the specific activity was 12.9 GBq/μmol). On day 2, first, the ABH2 was added to the blocking group at a concentration 50 times the concentration of the substance ^{99m}Tc-ABH2 in the non-blocking group, and then the ^{99m}Tc-ABH2 which was the same as that in the non-blocking group was added to the blocking group. The 24-well plate was left to stand at 37° C. for 1 h and washed with ice-cold PBS, and the radioactivity of cell binding was measured using a multi-channel γ spectrometer after digestion of the cells with 0.1 N NaOH; the counts of radioactivity of binding in the blocking group were subtracted from those in the non-blocking group to obtain counts of radioactivity of specific binding. Kd values were determined by GraphPad Prism5 software.

The detection results of the labeling rate, the radiochemical rate, the in vitro stability and the biological binding activity are shown in Table 2 below.

TABLE 2

| | | Concentration | | | | Biological | |
|---|---|---|---|---|---|---|---|
| Experimental | Concentration | of impurity | Labeling | Radiochemical | In vitro | binding | |
| Example | of protein | protein | rate | purity | stability | activity | Note |
| 2 | 0.24 g/L | 0.01 g/L | 99% | 100% | 98% | 1.0 nM | The amount |
| | | | | | | | of impurities |
| | | | | | | | is below |
| | | | | | | | detection |
| | | | | | | | limit |
| 3 | 0.24 g/L | 0.01 g/L | 84% | 95% | 94% | 1.7 nM | The amount |
| | | | | | | | of impurities |
| | | | | | | | is below |
| | | | | | | | detection |
| | | | | | | | limit |
| 4 | 0.24 g/L | 0.01 g/L | 94% | 99% | 97% | 1.0 nM | The amount |
| | | | | | | | of impurities |
| | | | | | | | is below |
| | | | | | | | detection |
| | | | | | | | limit |
| 5 | 0.24 g/L | 0.01 g/L | 67% | 96% | 96% | 1.5 nM | The amount |
| | | | | | | | of impurities |
| | | | | | | | is below |
| | | | | | | | detection |
| | | | | | | | limit |
| 6 | 0.24 g/L | 0.01 g/L | 90% | 96% | 95% | 1.3 nM | The amount |
| | | | | | | | of impurities |
| | | | | | | | is below |
| | | | | | | | detection |
| | | | | | | | limit |
| 7 | 0.024 g/L | 0.01 g/L | 46% | 90% | 89% | 1.2 nM | The amount |
| | | | | | | | of impurities |
| | | | | | | | is below |
| | | | | | | | detection |
| | | | | | | | limit |
| 8 | 3.12 g/L | 0.13 g/L | 98% | 98% | 97% | 2.5 nM | The amount |
| | | | | | | | of impurities |
| | | | | | | | is above |
| | | | | | | | detection |
| | | | | | | | limit |
| 9 | 0.24 g/L | 0.01 g/L | 95% | 99% | 97% | 1.1 nM | The amount |
| | | | | | | | of impurities |
| | | | | | | | is below |
| | | | | | | | detection |
| | | | | | | | limit |
| 10 | 0.24 g/L | 0.01 g/L | 45% | 69% | 66% | 3.3 nM | The amount |
| | | | | | | | of impurities |
| | | | | | | | is below |
| | | | | | | | detection |
| | | | | | | | limit |

The impurities IPTG, ampicillin, DTT, TCEP and EDTA were detected by HPLC.

The above examples only illustrate several embodiments of the present disclosure for the purpose of specific and detailed description, but should not be construed as limiting the scope of the present disclosure. It should be noted that various changes and modifications can be made by those of ordinary skills in the art without departing from the spirit of the present disclosure, and these changes and modifications are all within the scope of the present disclosure. Therefore, the protection scope of the present disclosure should be determined by the appended claims.

1. An HER2 affibody ^{99m}Tc labelling composition, comprising: HEPES, sodium glucoheptonate, vitamin C, cysteine, stannous chloride and an HER2 affibody.

2. The composition according to claim 1, wherein the HEPES has a concentration of 2-30 mmol/L, the sodium glucoheptonate has a concentration of 5-40 mmol/L, the vitamin C has a concentration of 2-30 mmol/L, the cysteine has a concentration of 5-40 mmol/L, the stannous chloride has a concentration of 0.1-5 mmol/L, and the HER2 affibody has a concentration of 0.01-0.6 mmol/L, and the composition has a pH of 5.0-7.5.

3. The composition according to claim 2, wherein the HEPES has a concentration of 5-20 mmol/L, the sodium glucoheptonate has a concentration of 10-30 mmol/L, the vitamin C has a concentration of 5-20 mmol/L, the cysteine has a concentration of 10-30 mmol/L, the stannous chloride has a concentration of 0.2-2 mmol/L, and the HER2 affibody has a concentration of 0.03-0.3 mmol/L, and the composition has a pH of 6.0-7.0.

4. The composition according to claim 2, wherein the HEPES has a concentration of 10 mmol/L, the sodium glucoheptonate has a concentration of 20 mmol/L, the vitamin C has a concentration of 10 mmol/L, the cysteine has a concentration of 20 mmol/L, the stannous chloride has a concentration of 0.4 mmol/L, and the HER2 affibody has a concentration of 0.05 mmol/L, and the composition has a pH of 6.6.

5. The composition according to claim 4, the HER2 affibody has GGGC at carboxyl terminus and HEHEHE at amino terminus.

6. An imaging agent comprising the HER2 affinity ^{99m}Tc labelling composition according to claim 1.

7. An imaging agent product, comprising the HER2 affibody ^{99m}Tc labelling composition according to claim 1.

8. The imaging agent product according to claim 7, wherein the imaging agent is a PET or SPECT imaging agent and the product is a separate agent or a kit.

9. A method for preparing an HER2 affibody ^{99m}Tc labelling composition, comprising the following steps: adding 500-20000 μCi of ^{99m}Tc into the HER2 affibody ^{99m}Tc labelling composition, and reacting at room temperature for 5-60 min under sealed conditions.

10. The method according to claim 9, wherein the ^{99m}Tc is added in an amount of 2000-10000 μCi, preferably 5000 μCi; reaction time is 10-15 min.
